(19) United States
(12) Patent Application Publication
(10) Pub. No.: US 2019/0341140 A1
(43) Pub. Date: Nov. 7, 2019

(54) ENTERPRISE MOBILE, CLOUD BASED HEALTH CARE COMPLIANCE SYSTEM

(71) Applicants: Ilan Nachmany, Closter, NJ (US); Sumer Mathur, Sterling, VA (US)

(72) Inventors: Ilan Nachmany, Closter, NJ (US); Sumer Mathur, Sterling, VA (US)

(73) Assignee: Healthcare Facility Compliance Corp., Englewood, NJ (US)

(21) Appl. No.: 16/184,891

(22) Filed: Nov. 8, 2018

Related U.S. Application Data
(60) Provisional application No. 62/583,453, filed on Nov. 8, 2017.

(51) Int. Cl. G16H 40/20; G06Q 10/06; G06Q 30/00

(52) U.S. Cl. CPC G16H 40/20; G06Q 30/018; G06Q 10/0635

(57) ABSTRACT

An invention is provided for assuring ongoing health care facility compliance. The invention includes generating inspection notifications for a plurality of inspection points of element of performance (EP) data stored in a database, wherein the EP data indicates a particular requirement of a health care facility environment standard, and each inspection point indicates a requirement of the EP data and provides a method of assigning risk scores to each of inspection points and in case of failure of that inspection point, ability to determine action options based on risk score. Inspections for are performed and the results recorded for each inspection point, wherein each result indicates whether the inspection point passes or fails a related requirement of the inspection point. When a result indicates inspection point(s) failed, one or more work order and notification relate to the failed inspection points are generated and sent to the work order system. The generated work orders are then monitored until all work orders related to the EP data are completed, and inspections for the EP data are reinitiated when the all work orders related to the EP data are completed. If needed the invention automatically produces data for Interim Life Safety Measures.

### CROSS REFERENCE TO RELATED APPLICATIONS

This application claims the benefit of U.S. Provisional patent application having Ser. No. 62/583,453, filed on Nov. 8, 2017, and entitled “Enterprise Mobile, Cloud Based Health Care Compliance and Risk Assessment System,” which is hereby incorporated by reference in its entirety.

### BACKGROUND OF THE INVENTION

### 1. Field of the Invention

This invention relates generally to health care facility compliance, and more specifically, to an enterprise mobile, cloud based health care compliance and risk assessment systems and methods.

### 2. Description of the Related Art

To improve healthcare quality, organizations have been developed to inspect and accredit health care facilities. Examples of such organizations include the Centers for Medicare & Medicaid Services (CMS) or accredited independent, not-for-profit organizations; i.e., The Joint Commission (TJC), Det Norske Veritas (DNV), Center for Improvement in Healthcare Quality (CIHQ) and Healthcare Facilities Accreditation Program (HFAP). For example, the Joint Commission helps to continuously improve health care for the public by evaluating health care organizations, and accredits and certifies more than 21,000 health care organizations and programs in the United States, including hospitals and health care organizations that provide ambulatory and office-based surgery, behavioral health, home health care, laboratory and nursing care center services. This organization dominates the market regulating 15,716 healthcare facilities in the United states, 3959 Hospitals, 848 Nursing care Center 363 Critical Access Hospitals 1941 Ambulatory Care 3017 Behavioral Health 5298 Home Care and 290 Office Based Surgery, a total of 78% of the US healthcare facilities.

Hospitals depend to varying degrees on receipt of Federal funds to meet their increasing budget needs. Along with partial Federal financing of hospitals, specific Federal requirements have been introduced requiring hospitals to demonstrate compliance in order to receive Medicare/Medicaid funding.

To demonstrate compliance, hospitals were initially given two options by Congress: Either submit to a Federal audit, or achieve accreditation from The Joint Commission (originally known as The Joint Commission on Accreditation of Hospitals, and later as The Joint Commission on Accreditation of Healthcare Organizations). Passing an audit by either of these two organizations would give a hospital “Deemed Status” for a 3-year period which would enable them to collect Medicare/Medicaid funds while they hold this status. Not obtaining or loosing this status would prohibit them from collecting this funding while they were not in “Deemed Status”.

Over the years the healthcare environment has gotten far more complex from a regulatory and technology standpoint as well as a medical and business standpoint. As this has occurred, the number of regulations has increased dramatically; the regulations themselves have become far more complex; documentation requirements have greatly increased; yet due to cost constraints, spending on programs and staffing has been continually cut by the healthcare providers.

From a regulatory standpoint as the result of survey findings, the Physical Environment has been at the top of the list in the past four decades relative to creation of new requirements, enforcement of requirements, and the impact of adverse requirements to an organization's ability to collect Medicare/Medicaid funds.

Specifically related to The Joint Commission, the Physical Environment standards have progressed to a point where there are now 3 relevant groups (chapters) of standards: Environment of Care; Life Safety Code; and Emergency Management, which between them represent 49 actual standards, with 443 specific elements of performance (EP's). Many, if not most, of those 443 EP's have multiple aspects and data points that must be managed and documented to ensure compliance. Of these 443 EP's, 77 are direct impact standards which can adversely affect an organization outcome and 2 are situational decision rules which can cause a survey to end immediately in a conditional or tentative non-accreditation.

The recent history of the industry is that in the last 10 years the number of findings in these areas greatly increased, largely due to increased and more frequent regulation changes, CMS directing the accrediting bodies to survey more aggressively; and surveyors have been given better training in these areas.

In view of the forgoing, there is a need for a system that provides hospitals with the ability to assure ongoing compliance and reduce cost by replacing their current existing costly manual inspections.

### SUMMARY OF THE INVENTION

Broadly speaking, embodiments of the present invention address these needs by providing a system, method, and computer program that affords hospitals the ability to assure ongoing compliance and reduce cost by using a central repository and tracking solution that helps improve healthcare physical environment quality of service and provides for successful compliance outcomes.

In one embodiment, a method is disclosed for assuring ongoing health care facility compliance. The method includes generating inspection notifications for a plurality of inspection points of element of performance (EP) data stored in a database, wherein the EP data indicates a particular requirement of a health care facility environment standard, and each inspection point indicates a requirement of the EP data. Typically, inspection points can include an asset related to the EP data, a document related to the EP data, or an action to be taken related to the EP data. The method includes an approach of assigning risk scores to each of the inspection points itself. More specifically, the method includes assigning and storing in a database a risk assessment score to each inspection point, wherein the risk assessment score is a number indicating an importance and potential harm created when the inspection point fails inspection. Inspections are performed and the results recorded for each inspection point, wherein each result indicates whether the inspection point passes or fails a related requirement of the inspection point and tracks associated risk score. When a result indicates an inspection point fails, a determination is made as to a severity level of risk that results and an appropriate action to take based on the risk assessment scores of failed inspection points. Hence, risk assessment scores resulting from an individual asset's inspection, as well as a collective risk-state of others assets in the physical environment, can be used to determine a severity level of outcomes and determine actions. When a result indicates an inspection point fails, a work order and notification related to the failed inspection are also generated and sent to the work order system. The generated work orders are then monitored until all work orders related to the EP data are completed as indicated via the work order system. Thereafter, inspections for the EP data are reinitiated.

In one aspect, the method can include determining whether a failed inspection point result relates to a risk assessment item, which is an inspection point that requires risk analysis. A further determination can be made as to whether failed inspection points based on the EP data indicate an Interim Life Safety Measure (ILSM) should be issued and issuing an ILSM if required. In addition, the method can include finalizing an inspection report for the EP data and auto filing the inspection report in a binder related to the EP data.

A computer program embodied on a computer readable medium for assuring ongoing health care facility compliance is disclosed in a further embodiment of the present invention. The computer program includes computer instructions that generate inspection notifications for a plurality of inspection points of EP data stored in a database. As above, the EP data indicates a particular requirement of a health care facility environment standard, and each inspection point indicates a requirement of the EP data. The computer program also includes computer instructions that assign and store in a database a risk assessment score to each inspection point, wherein the risk assessment score is a number indicating an importance and potential harm created when the inspection point fails inspection. Also included in the computer program are computer instructions that record results of inspections of each inspection point, wherein each result indicates whether the inspection point passes or fails a related requirement of the inspection point and risk score of each inspection point failure. Computer instructions are further included that determine a severity level of risk that results and an appropriate action to take based on the risk assessment scores of failed inspection points when a result indicates an inspection point fails. Similar to above, computer instructions are also included that generate a work order and notification when a result indicates an inspection point fails and sends the work order to a work order system. The computer program also includes instructions that monitor generated work orders until all work orders related to the EP data are completed, and instructions that reinitiate inspections for the EP data when the all work orders related to the EP data are completed as indicated via the work order system.

Similar to above, the computer program can include instructions that determine whether a failed inspection point result relates to a risk assessment item, wherein a risk assessment item is an inspection point that requires risk analysis. In this aspect, instructions also can be included that determine whether failed inspection points of the EP data indicate an Interim Life Safety Measure (ILSM) should be issued and issue an ILSM if required. The computer program can include instructions that finalize an inspection report for the EP data and file the inspection report in a binder related to the EP data.

In this manner, embodiments of the present invention provide warnings and alarms when deadlines approach, give emergency notification to users when deadlines have not been met, and provide ongoing, comprehensive regulatory compliance reports to assist in the management of these programs. As a result, hospitals can begin a survey knowing required compliance activities have been completed on time and documented correctly, thus dramatically reducing the probability of being cited overall. Other aspects and advantages of the invention will become apparent from the following detailed description, taken in conjunction with the accompanying drawings, illustrating by way of example the principles of the invention.

### BRIEF DESCRIPTION OF THE DRAWINGS

The invention, together with further advantages thereof, may best be understood by reference to the following description taken in conjunction with the accompanying drawings in which:

FIG. 1 is a block diagram showing an enterprise mobile, cloud based compliance system to increase hospital facility management compliance and to improve management and maintenance of healthcare facilities, in accordance with an embodiment of the present invention;

FIG. 2 is a flowchart showing a method for assuring ongoing health care facility compliance, in accordance with an embodiment of the present invention; and

FIG. 3 is a flow chart showing a method for compliance and risk assessment, in accordance with an embodiment of the present invention.

### DETAILED DESCRIPTION OF THE PREFERRED EMBODIMENTS

Embodiments of the present invention provide a system that affords hospitals the ability to assure ongoing compliance and reduce cost by migrating from their current existing costly manual inspections to using a central repository and tracking solution that helps improve healthcare physical environment quality of service and provides for successful compliance outcomes. Thus, embodiments of the present invention schedule regulatory compliance activities around regulatory mandated timelines. Embodiments also provide warnings and alarms when deadlines approach, give emergency notification to users when deadlines have not been met, and provide ongoing, comprehensive regulatory compliance reports to assist in the management of these programs. As a result, hospitals can begin a survey knowing required compliance activities have been completed on time and documented correctly, thus dramatically reducing the probability of being cited overall.

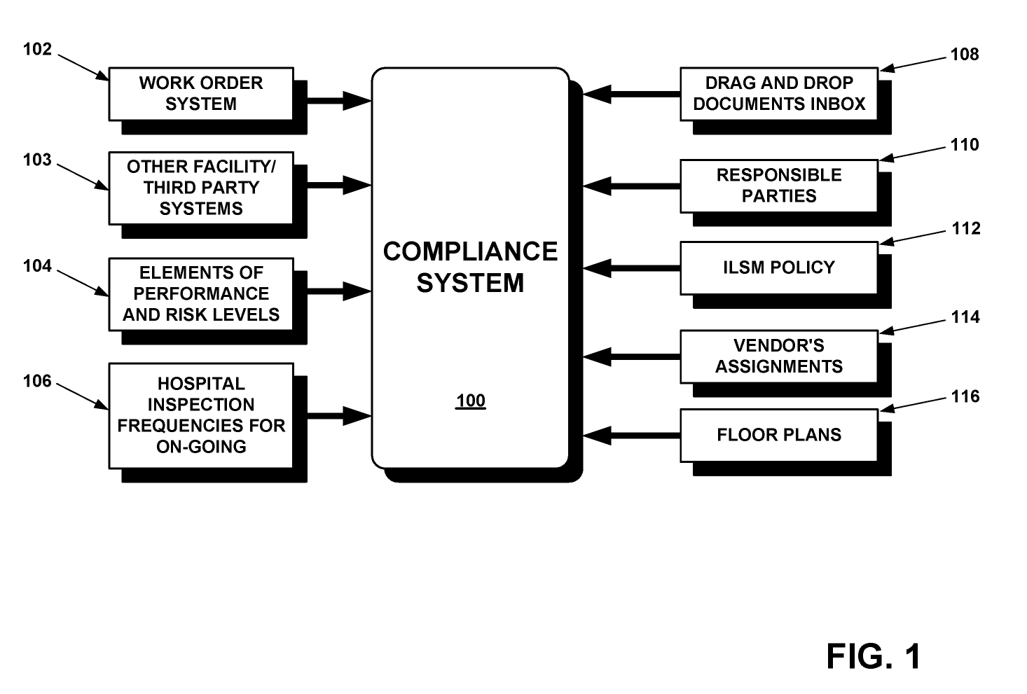

FIG. 1 is a block diagram showing an enterprise mobile, cloud based compliance system 100 to increase hospital facility management compliance and to improve management and maintenance of healthcare facilities, in accordance with an embodiment of the present invention. Embodiments of the present invention integrate with multiple sources of existing data to determine where the facility is in terms of compliance, what needs to be done, and provide the facility a schedule of what needs to be completed. To perform these functions, the compliance system 100 is initially set up to operate in a specific environment as discussed below.

Set up includes integrating the compliance system 100 with a facility's work order system 102, other facility/third party systems 103, providing elements of performance (EP) 104 and associated risk assessment scores based on the mandated frequencies and risk levels, providing the hospital's on-going inspection frequencies 106, and setting up an inbox 108 for documents emailed to the compliance system 100. In addition, responsible parties 110 are associated with each EP, an interim life safety measures policy 112 is provided to the compliance system 100, as are vendor assignments 114 and floor plans 116 for the associated facility.

Each standard from the Joint Commission has a plurality of elements of performance (EPs) to indicate what needs to be completed to satisfy the standard. An example EP could be “the fire doors need to be inspected once per year.” Embodiments of the present invention look not only at what frequency an asset should be inspected, but also how the inspection should be done (what should be the inspection points). If any of those inspection points is such that risk assessment is required embodiments of the present invention determine whether a life safety measure should be in place. As inspections are done, the associated documentation automatically files in the right place. In addition, if an inspection result requires a work order to be created, the compliance system 100 communicates with the work order system 102 to file a work order into the work order system 102 and then tracks the work order until it is completed. For actions performed by vendors outside the system, the vendors can email their report to the system and the report can be attached to a particular EP after going through inspection points related to acceptance of report in the system.

Embodiments of the present invention can be realized using an enterprise mobility system, wherein users utilize mobile assets, such as tablets and smart phones, to interact with the data a programs provided via the embodiments of the present invention. In general, the enterprise mobility nature of the embodiments of the present invention allow for increased productivity and decreased expenses for the facility.

In addition, embodiments of the present invention can be realized via Internet-based computing delivered to the facility's computers and devices through the Internet, often referred to as cloud based computing. This allows embodiments to be accessed and shared as virtual resources in a secure and scalable manner, which further supports enterprise mobility as described above. In some aspects, embodiments of the present invention can be accessed via the Internet, instead of a local hard drive. In these aspects, embodiments are delivered and utilized via the Internet. In this manner, the related infrastructure of the compliance system 100 can be maintained by a provider, instead of the health facility itself.

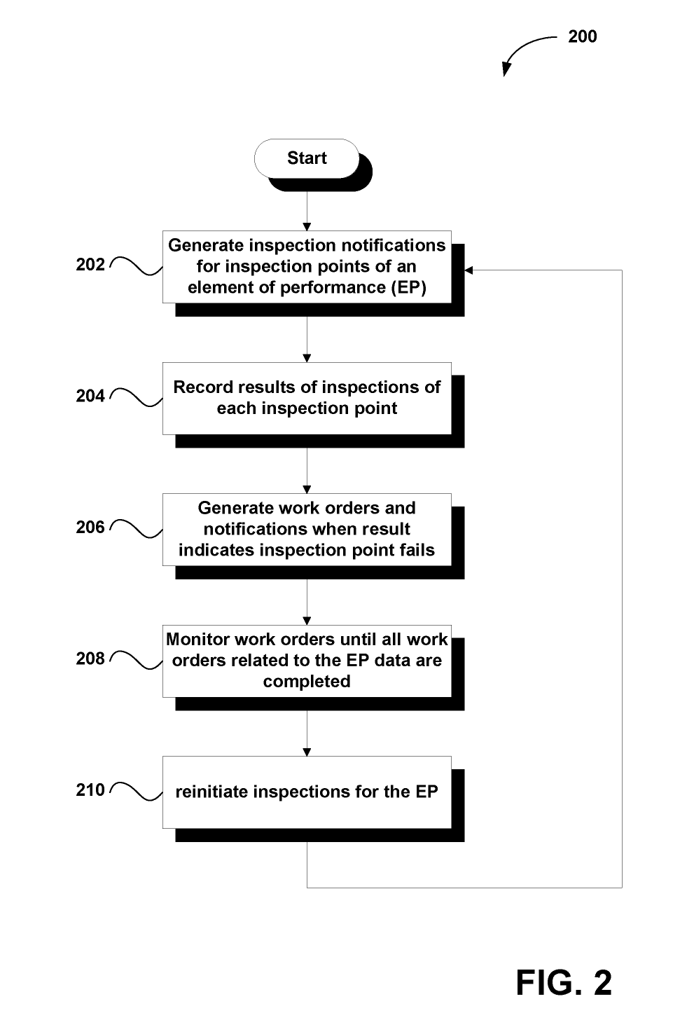

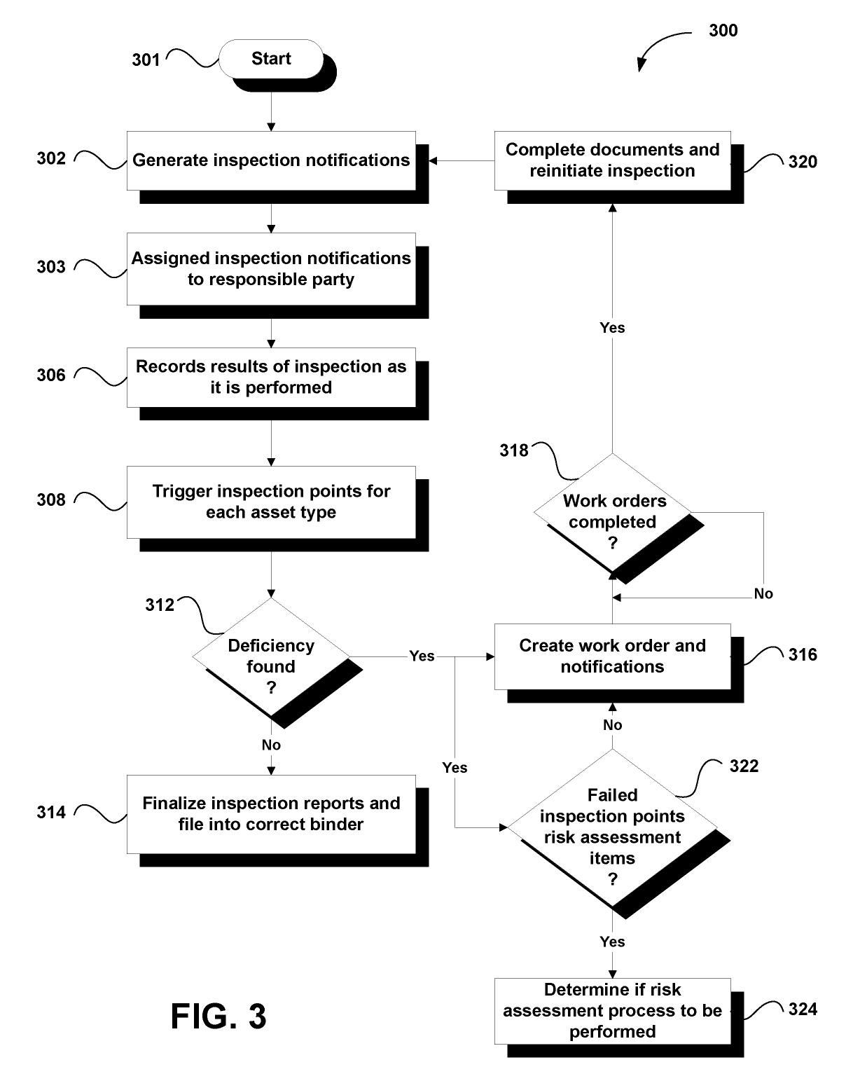

FIG. 2 is a flowchart showing a method 200 for assuring ongoing health care facility compliance, in accordance with an embodiment of the present invention. FIG. 2 discloses the core workflow for ensuring compliance with environmental standards for health care facilities for a single EP. This basic workflow is followed for each additional EP until all EPs of the standard are processed, as described in greater detail with reference to FIG. 3.

Referring to FIG. 2, inspection notifications are generated for a plurality of inspection points of EP data stored in a database, in operation 202. As noted previously, the EP data indicates a particular requirement of a health care facility environment standard, and each inspection point indicates a requirement of the EP data. The notifications are utilized to initiate inspections for each inspection point by the responsible party.

In operation 204, the results of the inspections of each inspection point are record to indicate whether the inspection point passes or fails. Each inspection point indicates a requirement of the respective EP. For example, an inspection point can be an asset inspection, a document, or an action to be taken. During this operation, the responsible party performs the inspection and the results of the inspection are recorded in the system.

Results indicating an inspection point fails generate a work order, which is sent to the work order system, and a notification that is sent to the responsible party, in operation 206. In this manner, failed inspection points are automatically addressed via the work order system. The work orders are monitored, in operation 208, until all work orders related to the EP data are completed, and thereafter, the inspections for the EP data are reinitiated, in operation 210.

In one embodiment, results indicating an inspection point fails also trigger a check for the risk assessment score on the inspection point. If risk score is above zero the method provides an option of time to resolve the deficiency up to a particular predefined number of hours. If unresolved the deficiency is marked and it moves to operation 206.

As noted previously, embodiments of the present invention follow the operations of FIG. 2 for each EP in the related standard. As a result, the user is required to address these EPs. As each EP is addressed additional Interim Life Safety measures may be issued to ensure patient and visitor safety. The entire process is described next in greater detail with reference to FIG. 3.

FIG. 3 is a flowchart showing a method 300 for compliance and risk assessment, in accordance with an embodiment of the present invention. In an initial operation 301, initialization and set up operations are performed. Initialization and set up operations can include, for example, integrating the compliance system with a facility's work order system, providing EP and associated risk assessment scores, providing the hospital's on-going inspection frequencies, associating responsible parties with each EP, providing an interim life safety measures policy, determining vendor assignments, and uploading facility floor plans.

In operation 302, inspection notifications are generated and, in operation 304, these notifications are assigned to in-house responsible party or an outside vendor. Based on a predetermined frequency, inspection notifications for EPs are generated by the compliance system. In one embodiment, the compliance system shows the user the standard requirements and the related elements of performance. Beside each EP the user is shown the mandatory frequency and the specific person assigned to each EP.

In operation 306, the system records the results of the inspection as it is performed. During the inspection, inspection points are triggered for each asset type, in operation 308. In addition, rounds and drills are recorded with respect to whether they have been performed. Embodiments of the present invention analyze each requirement of the JC to determine if the requirement relates to an asset inspection, a document, or if the requirement relates to a miscellaneous action that must be performed, such as filing rounds that performed every day.

A determination is then made as to whether a deficiency has been found in any inspection point, in operation 312. If no deficiency is found in any inspection point, the method 300 continues to operation 314. Otherwise, the method 300 branches to operation 316 and operation 322.

In operation 314, the inspection reports are finalized and filed into the correct binder. During this operation, the inspection reports are e-signed and the inspection points are reset to the next inspection date. In one embodiment, work from vendors, policies that must be reviewed, etc. are all stored in a repository that is used to show the user whether they have satisfied each requirement. If any requirement is not satisfied, the user can view into the repository and view what is upcoming, what is due, what is past due, where they are now, and how does that affect them in terms of the inspection that will be done by the JC.

In one embodiment, the compliance system has a plurality of binders. When documents are attached to the compliance system, the document is automatically filed into the appropriate binder. Items emailed to the system are sent to the inbox, as indicated in FIG. 1. From this inbox, email attachments can be attached to a particular EP. Each EP has its own document requirements indicating what the document needs to include in order to satisfy that EP. These requirements can include such aspects as, for example, a signature and/or a date. When a user attempts to attach a document to an EP, embodiments present a number of questions to the user based on what is required to satisfy the EP's document requirements. In order for the document to be accepted, the user must answer in the affirmative for each question.

If deficiencies are found in any inspection point, the method 300 branches to operation 316 and operation 322. In operation 316, a work order and notifications are created. As mentioned previously, embodiments of the present invention are initially integrated with a work order system. In operation 316, the compliance system communicates with the work order system to create one or more work orders to correct the deficiencies found during the inspection. The compliance system then monitors these work orders to determine when they are completed.

In operation 318, a determination is made as to whether the work orders are completed. If the work orders are not completed, the method 300 loops to another operation 318 as it continues to monitor the work orders. If the work orders are completed the method 300 continues to operation 320, where the documents are completed and the inspection is reinitiated.

In addition to performing operation 316, the method 300 branches to operation 322 when deficiencies are found in any inspection point. In operation 322, a decision is made as to whether any of the failed inspection points are risk assessment items. If none of the failed inspection points are risk assessment items, no additional actions are performed and the method continues to generate work orders and monitor them as discussed above with respect to operations 316 and 318. However, if any of the failed inspection points are risk assessment items, a risk assessment process 324 is performed.

During an inspection, if a deficiency is found in an inspection point that is a risk assessment item, the risk assessment process 300 is performed. In the compliance system, particular inspection points require risk analysis, and thus are given a score above zero based on the importance and the harm created when the inspection point fails inspection. Setting a risk assessment score to zero indicates the inspection point does not require risk analysis. Thus, in operation 324, the system determines whether a failed inspection point result relates to a risk assessment item, which is an inspection point that requires risk analysis.

In one embodiment, whenever an inspection point that relates to a risk assessment item fails inspection, the user is given a choice of whether to have “Time to Resolve” or to “Mark as deficiency.” When the user selects “Time to Resolve,” the user is given a predetermined amount of time as specified by the EP, for example four hours, to resolve the inspection point deficiency before the inspection point failure result is transferred to a required mandatory risk analysis. If the user resolves the deficiency within the predetermined time period, the inspection point failure is not transferred to risk analysis, otherwise the inspection point failure is transferred to risk analysis. If the user selects “Mark as deficiency,” the inspection point failure is immediately transferred to risk analysis. In risk analysis, a determination is made as to whether failed points of inspection of the EP data indicate an Interim Life Safety Measure (ILSM) should be issued and issuing an ILSM if required.

Although the foregoing invention has been described in some detail for purposes of clarity of understanding, it will be apparent that certain changes and modifications may be practiced within the scope of the appended claims. Accordingly, the present embodiments are to be considered as illustrative and not restrictive, and the invention is not to be limited to the details given herein, but may be modified within the scope and equivalents of the appended claims.

What is claimed is:

1. A method for assuring ongoing health care facility compliance, comprising:
generating inspection notifications for a plurality of inspection points of element of performance (EP) data stored in a database, wherein the EP data indicates a particular requirement of a health care facility environment standard, and wherein each inspection point indicates a requirement of the EP data;
assigning and storing in a database a risk assessment score to each inspection point, wherein the risk assessment score is a number indicating an importance and potential harm created when the inspection point fails inspection;
recording results of inspections of each inspection point, wherein each result indicates whether the inspection point passes or fails a related requirement of the inspection point;
generating a work order and notification when a result indicates an inspection point fails and sending the work order to a work order system, wherein the work order and notification relate to the failed inspection point;
determining a severity level of risk and an appropriate action to take based on the risk assessment scores of failed inspection points;
monitoring generated work orders until all work orders related to the EP data are completed; and
reinitiating inspections for the EP data when the all work orders related to the EP data are completed as indicated via the work order system.

2. A method as recited in claim 1, further comprising determining whether a failed inspection point result relates to a risk assessment item, wherein a risk assessment item is an inspection point that requires risk analysis.

3. A method as recited in claim 2, further comprising determining whether failed inspection points of the EP data indicate an Interim Life Safety Measure (ILSM) should be issued and issuing an ILSM if required.

4. A method as recited in claim 1, wherein the inspection point is an asset related to the EP data.

5. A method as recited in claim 1, wherein the inspection point is a document related to the EP data.

6. A method as recited in claim 1, wherein the inspection point is an action to be taken related to the EP data.

7. A method as recited in claim 1, further comprising finalizing an inspection report for the EP data and filing the inspection report in a binder related to the EP data.

8. A method for assuring ongoing health care facility compliance, comprising:
generating inspection notifications for a plurality of inspection points of element of performance (EP) data stored in a database, wherein the EP data indicates a particular requirement of a health care facility environment standard, and wherein each inspection point indicates a requirement of the EP data;
assigning and storing in a database a risk assessment score to each inspection point, wherein the risk assessment score is a number indicating an importance and potential harm created when the inspection point fails inspection;
recording results of inspections of each inspection point, wherein each result indicates whether the inspection point passes or fails a related requirement of the inspection point;
determining whether a failed inspection point result relates to a risk assessment item, wherein a risk assessment item is an inspection point that requires risk analysis;
determining a severity level of risk and an appropriate action to take based on the risk assessment scores of failed inspection points;
generating a work order and notification when a result indicates an inspection point fails and sending the work order to a work order system, wherein the work order and notification relate to the failed inspection point;
monitoring generated work orders until all work orders related to the EP data are completed;
determining whether failed inspection points of the EP data indicate an Interim Life Safety Measure (ILSM) should be issued and issuing an ILSM if required; and
reinitiating inspections for the EP data when the all work orders related to the EP data are completed as indicated via the work order system.

9. A method as recited in claim 8, wherein the inspection point is an asset related to the EP data.

10. A method as recited in claim 8, wherein the inspection point is a document related to the EP data.

11. A method as recited in claim 8, wherein the inspection point is an action to be taken related to the EP data.

12. A method as recited in claim 8, further comprising finalizing an inspection report for the EP and filing the inspection report in a binder related to the EP data.

13. A computer program embodied on a computer readable medium for assuring ongoing health care facility compliance, comprising:
computer instructions that generate inspection notifications for a plurality of inspection points of element of performance (EP) data stored in a database, wherein the EP data indicates a particular requirement of a health care facility environment standard, and wherein each inspection point indicates a requirement of the EP data;
computer instructions that assign and store in a database a risk assessment score to each inspection point, wherein the risk assessment score is a number indicating an importance and potential harm created when the inspection point fails inspection;
computer instructions that record results of inspections of each inspection point, wherein each result indicates whether the inspection point passes or fails a related requirement of the inspection point;
computer instructions that generate a work order and notification when a result indicates an inspection point fails and sends the work order to a work order system, wherein the work order and notification relate to the failed inspection point;
computer instructions that determine a severity level of risk and an appropriate action to take based on the risk assessment scores of failed inspection points;
computer instructions that monitor generated work orders until all work orders related to the EP data are completed; and
computer instructions that reinitiate inspections for the EP data when the all work orders related to the EP data are completed as indicated via the work order system.

14. A computer program as recited in claim 13, further comprising computer instructions that determine whether a failed inspection point result relates to a risk assessment item, wherein a risk assessment item is an inspection point that requires risk analysis.

15. A computer program as recited in claim 14, further comprising computer instructions that determine whether failed inspection points of the EP data indicate an Interim Life Safety Measure (ILSM) should be issued and issue an ILSM if required.

16. A computer program as recited in claim 13, wherein the inspection point is an asset related to the EP data.

17. A computer program as recited in claim 13, wherein the inspection point is a document related to the EP data.

18. A computer program as recited in claim 13, wherein the inspection point is an action to be taken related to the EP data.

19. A computer program as recited in claim 13, further comprising computer instructions that finalize an inspection report for the EP data and file the inspection report in a binder related to the EP data.

20. A computer program as recited in claim 13, wherein multiple failed inspection points are incorporated into a single work order.
